(19) United States
(12) Patent Application Publication
(10) Pub. No.: US 2009/0240228 A1
(43) Pub. Date: Sep. 24, 2009

(54) ADJUSTABLE ABSORBENT ARTICLE FOR IMPROVED FIT

(75) Inventors: Heather R. Nonnenmann, Warminster, PA (US); Thomas Bergin, Conshohocken, PA (US)

Correspondence Address: AMSTER, ROTHSTEIN & EBENSTEIN LLP, 90 PARK AVENUE, NEW YORK, NY, 10016, US

(73) Assignee: TYCO HEALTHCARE RETAIL SERVICES AG, Neuhausen am Rheinfall (CH)

(21) Appl. No.: 12/050,528

(22) Filed: Mar. 18, 2008

(51) Int. Cl. A61F 13/64; A61F 13/493

(52) U.S. Cl. 604/385.3; 604/392

(57) ABSTRACT

Apparatus and methods of manufacturing absorbent articles suitable for use as training pants or other hygiene products are provided. The absorbent article includes a chassis having a front section, a rear section, and a crotch section extending between the front section and the rear section. The front section is connected to the rear section to define a waist opening, leg openings, and a belly region positioned between the waist opening and the leg openings. The absorbent article also includes an absorbent core coupled to the crotch section of the chassis. An adjustable tensioner is coupled to the chassis and is positioned for snugging the chassis against a wearer of the absorbent article. The tensioner is configured to be adjusted by the wearer from a first tension to a second, selected tension.

### FIELD OF THE INVENTION

The present invention relates to absorbent articles such as diapers, training pants, protective underwear, adult diapers, and the like. More specifically, the invention relates to absorbent articles having a tensioner and elastic member configuration for improved fit.

### BACKGROUND OF THE INVENTION

Absorbent articles such as disposable diapers, training pants, protective underwear, adult diapers, and the like are known for their major function of absorbing and containing body exudates. Such articles are thus intended to prevent the soiling, wetting, or other contamination of clothing or other articles, such as bedding, that come in contact with the wearer. In the case of disposable training pants, they typically include a chassis with a liquid permeable topsheet, a liquid impermeable backsheet, and an absorbent core positioned between the topsheet and the backsheet. The chassis is configured for fitting the training pant about the wearer's waist, thereby allowing the training pant to be pulled up or down like traditional underwear.

While many developments have been made in the art of absorbent articles, there remains a need for further improvements in terms of at least one of improved fit, comfort, producibility, and visual appearance.

### SUMMARY OF THE INVENTION

In one aspect, the invention provides an absorbent article that includes a chassis having a front section, a rear section, and a crotch section extending between the front section and the rear section. The front section is connected to the rear section to define a waist opening, leg openings, and a belly region positioned between the waist opening and the leg openings. The absorbent article also includes an absorbent core coupled to the crotch section of the chassis. An adjustable tensioner is coupled to the chassis and positioned for snugging the chassis against a wearer of the absorbent article. The tensioner is configured to be adjusted by the wearer from a first tension to a second, selected tension in which the first tension is less than the second, selected tension.

In another aspect of the invention, the invention further provides an absorbent article including a chassis having a front section, a rear section, and a crotch section extending between the front section and the rear section. The front section is connected to the rear section to define a waist opening, leg openings, and a belly region positioned between the waist opening and the leg openings. The absorbent article also includes an absorbent core coupled to the crotch section of the chassis. At least one elastic member is coupled to each of the waist opening and the belly region, the elastic members being spaced apart from each other and being stretchable independent of one another. The elastic member coupled to the belly region has at least two elastic portions, in which the tension in one of the elastic portions differs from the tension in another one of the elastic portions.

In yet another aspect, the invention provides an absorbent article including a chassis having a front section, a rear section, and a crotch section extending between the front section and the rear section. The front section is connected to the rear section to define a waist opening, leg openings, a belly region positioned between the waist opening and the leg openings, and a rise extending from the leg openings to the waist opening. The absorbent article also includes an absorbent core coupled to the crotch section of the chassis. A tensioner is coupled to the chassis and is positioned for snugging the chassis against a wearer of the absorbent article. The absorbent article further includes means for reducing the rise of the chassis on the wearer defined in the belly region of the chassis at a location between the waist opening and the tensioner.

According to yet another embodiment, the invention provides a method of manufacturing an absorbent article. The method includes attaching an absorbent core to a chassis having a contiguous front, rear, and crotch sections, the crotch section extending between the front section and the rear section. A tensioner is attached to the chassis such that the tensioner is adjustable by the wearer from a first tension to a second, selected tension, in which the first tension is less than the second, selected tension.

In another embodiment, the invention provides a method of manufacturing an absorbent article including the step of attaching an absorbent core to a crotch section of a chassis at least one elastic member is coupled to each of a waist opening and a belly region of the chassis and the elastic members are spaced from each other such that the elastic members are stretchable independent of one another. The elastic member coupled to the belly region are configured the to have at least two elastic portions such that the tension in one of the elastic portions differs from the tension in another one of the elastic portions.

In yet another embodiment, the invention includes a method of using an absorbent article. The method includes the step of positioning the absorbent article on a wearer. At least one adjustable tensioner of the absorbent article is adjusted, thereby snugging the fit of the absorbent article on the wearer from a first tension to a second, selected tension in which the first tension is less than the second, selected tension.

### BRIEF DESCRIPTION OF THE DRAWINGS

The invention is best understood from the following detailed description when read in connection with the accompanying drawings, with like elements having the same reference numerals. This emphasizes that according to common practice, the various features of the drawings are not drawn to scale. On the contrary, the dimensions of the various features are arbitrarily expanded or reduced for clarity. Included in the drawings are the following figures:

FIG. 1 is a perspective view of an absorbent article according to an exemplary embodiment of the invention;

FIGS. 2A and 2B are perspective views of a tensioner, such as a drawstring, of the absorbent article shown in FIG. 1 according to aspects of the invention;

FIG. 3 is a perspective view of an absorbent article according to another embodiment of the invention;

FIG. 4 is a perspective view of an absorbent article having tensioners, such as drawstrings, attached to the sides of the absorbent article according to yet another embodiment of the invention;

FIGS. 5A and 5B are perspective views of a tensioner of the absorbent article shown in FIG. 4 according to aspects of the invention;

FIG. 6 is a perspective view of an absorbent article illustrating elastic members coupled to a belly region or waist portion of the absorbent article according to yet another embodiment of the invention;

FIG. 7 is an enlarged plan view of the belly region or waist portion of the absorbent article shown in FIG. 6 according to aspects of the invention; and

FIG. 8 is a perspective view of an absorbent article according to another exemplary embodiment of the invention.

### DETAILED DESCRIPTION OF THE INVENTION

The invention will next be described with reference to the figures. Such figures are intended to be illustrative rather than limiting and are included herewith to facilitate the explanation of exemplary features of embodiments of the present invention. The figures are not to scale, and are not intended to serve as engineering drawings.

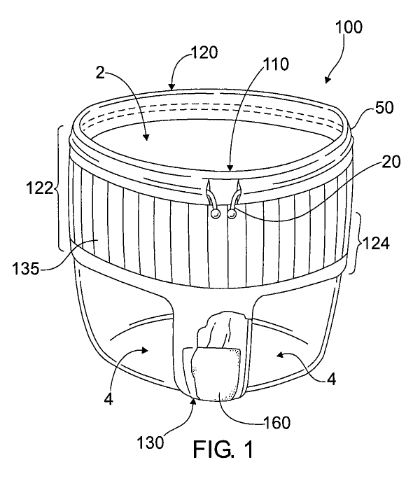

Referring generally to the drawings (FIGS. 1-8), in accordance with an exemplary embodiment, the invention provides an absorbent article 100, 200, 300, 500, 800 having a chassis that includes a front section 110, 210, 310, 510, 810; a rear section 120, 220, 320, 520, 820; and a crotch section 130, 230, 320, 520, 820 extending between the front section 110, 210, 310 510, 810 and the rear section 120, 220, 320, 520, 820. The front section 110, 210, 310, 510, 810 is connected to the rear section 120, 220, 320, 520, 820 to define a waist opening 2, leg openings 4, and a belly region 122, 222, 322, 522, 822 of the chassis. The chassis includes an absorbent core 160, 260, 360, 560, 860 coupled to the crotch section 130, 230, 320, 520, 820. The chassis also includes an adjustable tensioner 20, 40, such as a drawstring, coupled to the chassis. The tensioner 20, 40 is configured to be adjusted by the wearer from a first tension to a second, selected tension to releaseably contract or expand the waist opening 2 or leg openings 4 of the chassis.

According to another embodiment, the invention provides an absorbent article 500 comprising a chassis having a front section 510, a rear section 520, and a crotch section 530 extending between the front section 510 and the rear section 520. The front section 510 is connected to the rear section 520 to define a waist opening 2, leg openings 4, and a belly region 522 positioned between the waist opening 2 and the leg openings 4 of the chassis. The absorbent article 500 also includes an absorbent core 560 coupled to the crotch section 530 of the chassis. At least one elastic member 525a, 525b is coupled to each of the waist opening 2 and the belly region 522 of the chassis. The elastic members 525a, 525b are spaced apart from each other and stretchable independent of one another. The elastic member 525b coupled to the belly region 522 has at least two elastic portions 9a, 9b, wherein the tension in one of the elastic portions 9a, 9b differs from the tension in another one of the elastic portions 9a, 9b.

Referring now to the drawings in detail, FIG. 1 depicts an absorbent article 100 according to one exemplary embodiment of the present invention. Absorbent article 100 includes a chassis having a front section 110, a rear section 120, and a crotch section 130. The front section 110 is configured to be positioned against the anterior or front end of a wearer and the rear section 120 is configured to be positioned on the posterior or back end of a wearer. When worn by an individual standing upright, the crotch portion 130 of the chassis forms the lowest point on absorbent article 100. The front section 110 is connected to the rear section 120 to define a waist opening 2 and leg openings 4 of the absorbent article 100. According to an embodiment, edges of the front section 110 may be joined to edges of the rear section 120 to connect the front section 110 to the rear section 120. For example, suitable techniques for joining the front section 110 to the rear section 120 include adhesive bonding, heat sealing, ultrasonic bonding, stitching or the like.

As further illustrated in FIG. 1, absorbent article 100 also includes an absorbent core 160. Absorbent core 160 may be of any shape, but is typically a rectangular member that is centered in the chassis and positioned in crotch section 130. According to an embodiment, absorbent core 160 is interposed between a liquid permeable topsheet and a liquid impermeable substrate layer (not shown). In use, the topsheet is arranged to face toward the body of the user (i.e. against the skin of the wearer) and the substrate layer is arranged facing away from the skin of the wearer. Absorbent core 160 is configured to receive liquid through the topsheet so that liquid is trapped between the topsheet and liquid impermeable substrate layer thereby preventing soiling of the wearer's clothes.

In order to enable liquid to quickly and efficiently pass through the topsheet and into the absorbent core 160 for trapping therein, the topsheet is preferably liquid permeable. In particular, the topsheet may be selected from a variety of textile-like films and fabrics. Suitable fabrics include non-woven materials that are pervious to liquid, soft and pliable. Preferred non-woven materials include spun-bonded polypropylene, spunbonded polyethylene, and thermally bonded webs of staple fibers, preferably polypropylene shape or sheath/core bi-component fibers having a core of polyester or polypropylene and a sheath of polyethylene.

Absorbent core 160 may be made of any suitable absorbent material, as well as combinations of different types of absorbent materials. For example, the absorbent core 160 may be formed of a mixture of pulp fluff and superabsorbent polymer (SAP) wrapped in a liquid permeable tissue wrap (not shown). Examples of SAP include polyacrylamides, polyvinyl alcohol, polyacrylates, various grafted starches, and the like. A desired super absorbent material is a cross-linked polysodium acrylate, which can be purchased from BASF Corporation of Portsmouth, Va., under the trademark ASAP® 2260. The super absorbent materials can be in various geometric forms, such as various shaped particles, fibers, foams, and layers. The fluff and SAP are present, for example, in a ratio such as about 11 grams of SAP to 16 grams of fluff for a size 4 diaper, and have a core density, for example, in a range such as about 0.14 to 0.22 grams per cubic centimeter. The amount of each absorbent material and SAP/fluff ratio depends on factors including the size of the article, e.g., “Small”, “Medium”, “Large” or “Extra Large.”

Absorbent core 160 may be of any shape and may be a single, integral absorbent structure, or can comprise a plurality of individual separate absorbent structures and/or absorbent materials that are operably assembled together. It may also include an air-laid non-woven web that contains super-absorbent particles and/or super-absorbent fibers, polymeric binder and cellulose pulp fibers.

As seen with respect to the embodiment of the absorbent article 100 shown in FIG. 1, absorbent article 100 also includes a tensioner 20, such as a drawstring or other flexible member, coupled to the chassis. Tensioner 20 may be coupled to absorbent article 100 in any number of locations, including but not limited to any combination on the periphery of the chassis. For example, tensioner 20 may be coupled to any location on the waist portion 122 of absorbent article 100 such as the circumference around waist opening 2, leg openings 4, and/or belly region 122. In an embodiment, tensioner 20 may be coupled to side seams or portions of absorbent article 100 where edges of the front section 110 connect to the edges of the rear section 120. Tensioner 20 may also be located on either the inside (body facing) or outside of the absorbent article 100 depending on the manufacturing process. A tensioner 20 that is located on the outside of absorbent article 100, for example, may facilitate access to the tensioner 20 to tighten or loosen the fit of absorbent article 100 on the wearer.

As will be described in detail below, tensioner 20 can have different lengths and sizes to accommodate the waist opening 2, leg openings 4, and/or belly region 122 of the absorbent article 100. For example, an absorbent article 100 having a size of “Small” may include a tensioner 20 having a length that accommodates adult waist sizes of 28-32 inches. It is contemplated that other lengths may also be used to accommodate the circumference of the waist opening 2 and leg openings 4 of absorbent articles 100 for children and infants. Tensioner 20 may be fabricated from any suitable material including, but not limited to nylon, polyester, cotton, or other natural or synthetic material safe for use with contact to human skin. It is contemplated that other materials for tensioner 20 may also be used.

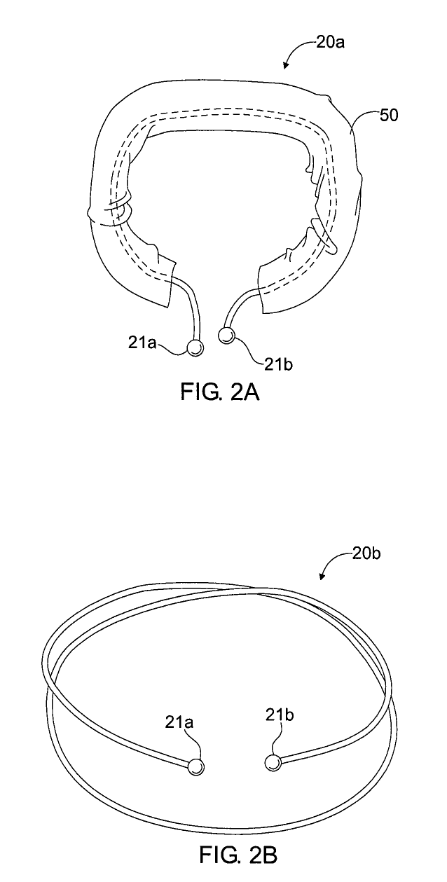

Referring now to FIGS. 1, 2A, and 2B, embodiments and operation of the tensioner 20 shown in FIG. 1A is illustrated. As shown in FIGS. 1 and 2A, tensioner 20a may be enclosed within a sheath 50, such as that attached to the circumference of the belly region 122 or waist portion 124 of the absorbent article 100. According to an exemplary embodiment, tensioner 20a is a single loop that has terminal ends 21a, 21b that extend from the circumference of the chassis. When the ends 21a, 21b of tensioner 20a are pulled, the circumference of the single loop decreases to cinch a portion of absorbent article 100. For example, when tensioner 20a is positioned around the belly region 122 of the absorbent article 100, pulling the ends 21a, 21b of tensioner 20a causes the belly region 122 to tighten, thereby decreasing the circumference of waist opening 2 and securing absorbent article 100 on the wearer. According to an embodiment, the ends 21a, 21b of tensioner 20a may be tied together or held in place to maintain the desired fit of absorbent article 100 on the wearer. Alternatively, the ends 21a, 21b of tensioner 20a may be loosened to allow the wearer to expand the waist opening 2 or leg openings 4 to remove or reposition absorbent article 100.

According to an embodiment, absorbent article 100 includes an elastic portion 135 coupled to the chassis. Elastic portion 135 is stretchable in at least a direction of the waist opening 2 so belly region 122 or waist portion 124 may be expanded to accommodate the belly/waist of the wearer. Thus, elastic portion 135 and tensioner 20 are adjustable to improve the fit of absorbent article 100 on the wearer. As will be described in further detail below, elastic portion 135 may include elastic strands (not shown) that provide the stretchability of the belly region 122 or waist portion 122.

FIG. 2B illustrates an embodiment of a tensioner 20b having a double loop. As described above, according to an exemplary embodiment, when ends 21a, 21b of the tensioner 20b is pulled, the circumference of the double loop decreases, thereby drawing belly region 122 of absorbent article 100 closer to the wearer. A tensioner 20b having a double loop provides additional support strength to keep absorbent article 100 in place on the wearer. In other embodiments, it is contemplated that tensioner 20b may be coiled in any number of loops to provide sufficient strength to support the weight of absorbent article 100.

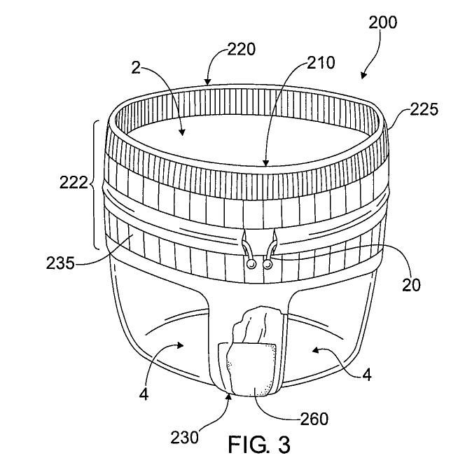

Referring now to FIG. 3, a perspective view of an absorbent article 200 according to another embodiment is illustrated. As described above, absorbent article 200 includes a chassis having a front section 210, a rear section 220, and a crotch section 230. The front section 210 is connected to the rear section 220 to define a waist opening 2 and leg openings 4 of the absorbent article 200. An absorbent core 260 is positioned in the crotch section 230 of the chassis and receives liquid from the body facing side of crotch section 230.

Absorbent article 200 includes an elastic member 225 or elastic band coupled to a upper portion of the belly region 222, such as around the waist opening 2. Elastic member 225 may be stretchable along the waist opening 2 and has a tension greater than elastic portion 235. Thus, elastic member 225 may support a portion of absorbent article 200 on a wearer. In the embodiment illustrated in FIG. 3, tensioner 20 is coupled to belly region 222 at a location below elastic member 225. Absorbent article 200 may be worn so the elastic member 225 may be pulled around the belly of the wearer and elongate member 20 may be cinched around the hips. According to another embodiment, belly region 222 may be folded over the inside (body facing) or outside of absorbent article 200 so the elastic member 225 is positioned below tensioner 20. It is contemplated that belly region 222 may be folded over itself in any configuration so a person can adjust the fit of absorbent article 200. It is also contemplated that any number of tensioners 20 may be coupled to absorbent article 200 to provide additional adjustability for a wearer.

According to another embodiment, leg portions of the absorbent article 100 adjacent leg openings 4 may include elastic portions 235 and/or elastic members 225. In this configuration, elastic portions 222 and/or members 225 positioned around the circumference of leg openings 4 provide additional support to secure absorbent article 200 so that it does not droop or sag on a wearer.

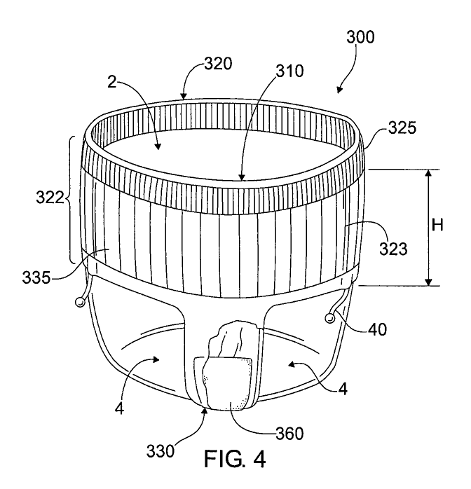

Referring now to FIG. 4, a perspective view of an absorbent article 300 is illustrated. According to an exemplary embodiment, tensioners 40 are coupled to sides of waist portion 322. Tensioners 40, for example, may be connected to side seams or portions of the front section 310 or rear section 320. The tensioners 40 are adjustable such that pulling tensioner 40 decreases the height H of belly region 322. For example, tensioner 40 may be used to pull up the sides of absorbent article 300 adjacent leg openings 4. This provides better fit of the lower portion of the absorbent article 300 on a wearer.

As described above, absorbent article 300 includes an elastic portion 335 and member 325 coupled to waist portion 322. Elastic portion 335 and member 325 assist stretching the circumference of waist opening 2 to accommodate a wearer. Belly region 322 may be folded over itself to further adjust the fit of absorbent article 300. Alternatively, side portion 323 may be extended to cover a larger leg area by pulling tensioner 40. Thus, side portions 323 of the absorbent article 300 may be adjustable for better comfort to a wearer. As will be described in detail below, tensioners 40 may enclosed in sheaths that are coupled to the belly region 322 and/or side portion 323 of the absorbent article 300.

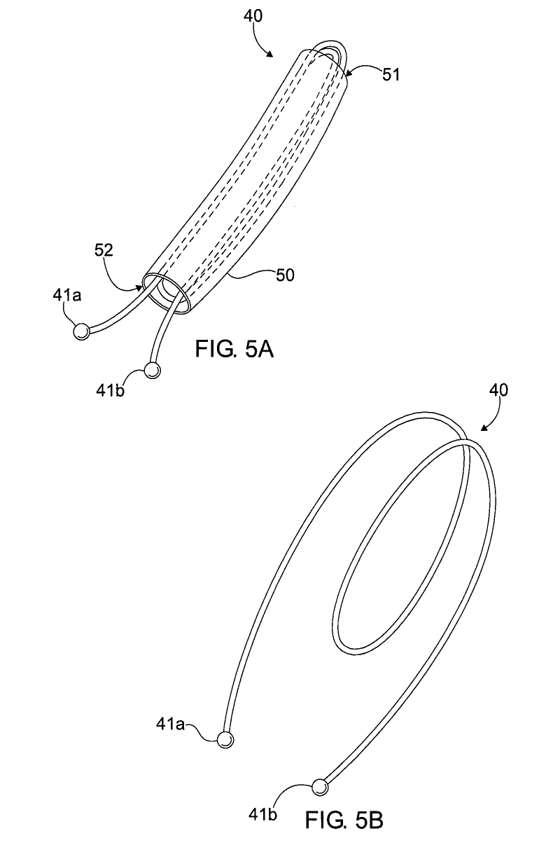

Referring now to FIGS. 4, 5A, and 5B, the tensioners 40 shown in FIG. 4 are illustrated. According to an exemplary embodiment, tensioner 40 is positioned within a sheath 50 that may be attached to absorbent article 300. When an end portion 41a, 41b of tensioner 40 is pulled, a portion of the sheath 50 (such as the proximal 51 and distal ends 52 of the sheath 50) and/or a belly region 322 of the absorbent article 300 shortens, thereby decreasing the height of the absorbent article 300. For example, when absorbent article 300 is worn, pulling the ends 41a, 41b of tensioner 40 causes the leg openings 4 to shorten, thereby cinching the leg openings 4 closer to crotch section 330. Alternatively, the tensioners 40 can be un-tensioned to return the side portions 323 of the absorbent article 300 to is original height.

According an exemplary embodiment, the tensioners 40 can be secured to the absorbent article 300 using a nonwoven or other suitable material formed into a single or double chamber sheath 50 adhered to the absorbent article 300, thus facilitating shortening of the absorbent article 300 height. In another exemplary embodiment, tensioner 40 may be removed from the sheath 50 or the ends 41a, 41b can be left in place when absorbent article 300 is worn (e.g., so that the side portion 323 is not cinched). For example, in an embodiment where the tensioner is removed, elastic portion 325 or member 325 of the absorbent article 300 may be used to hold the absorbent articled 300 against the skin of the wearer. In an embodiment, in which the ends 41a, 41b of the tensioner are tied in place, however, the weight of the absorbent article 300 may be supported by the tension applied by the tensioner 40. As shown in FIG. 5B, accordingly to an exemplary embodiment, tensioner 40 may have a double loop to provide additional support strength and pulling ends 41a, 41b of the tensioner 40 shortens or lengthens the side portions 323 of the absorbent article 300.

Referring now to FIG. 6, an absorbent article 500 having two elastic members 525a, 525b coupled to belly region 522 and/or waist portion 524 is illustrated. Elastic members 525a, 525b are spaced apart from each other so a chassis region 527 of lower stretchability exists between the elastic members 525a, 525b. Alternatively, the chassis region 527 may have a high tension and the elastic members 525a, 525b may have a lower tension area. Accordingly to an exemplary embodiment, absorbent article 500 may also include a tensioner 20, 40 (FIGS. 2A, 2B, 5A, 5B) to enhance the fit of the absorbent article 500 on a wearer.

According to another exemplary embodiment, elastic portions 525a, 525b may both have a high tension to provide better fit. For example, an adult sized absorbent article 500 having a larger mass than a child sized training pant may have higher tension elastic members 525a, 525b to support the weight of the larger sized article. Elastic member 525a may be positioned above the waist, such as the belly area, and elastic member 525b may be positioned on the waist, thereby providing better fit and comfort for a wearer. According to an exemplary embodiment, elastic members 525a, 525 may have at least two elastic portions 9a, 9b that have different tensions to provide the tension to the belly region 522 or waist portion 524 of the absorbent articled.

In another embodiment, absorbent article 500 may be folded over along the belly region 522 or waist portion 524 to reduce the rise of the absorbent article 500 to a more comfortable height and minimize the possibility of absorbent article 500 from slipping. For example, elastic member 525a may be folded above or below elastic member 525b to decrease the height of the absorbent article 500.

As will be described in further detail below, in an exemplary embodiment, elastic portion 525a, 525b includes lycra or other stretchable elastic strands of various weight and thicknesses (e.g., decitex) to provide different tensions and/or regions of the absorbent article 500 with different or similar compression. For example, elastic tensions may range from 200 to 380% elongation. In another embodiment, the area directly above elastic portion 525b which may be positioned at the waist may include perforations to facilitate tearing, trimming, or separation of a portion of the absorbent article 500.

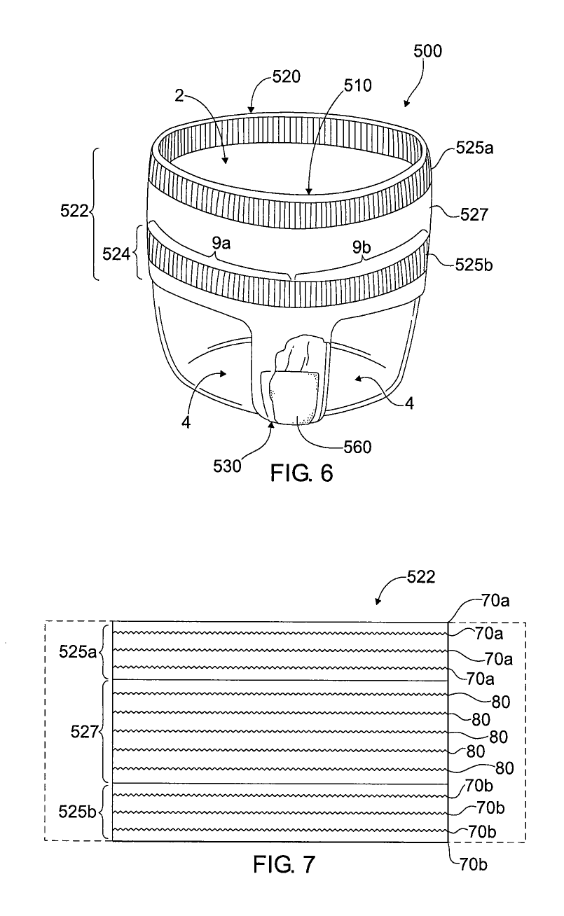

Referring now to FIG. 7, an enlarged section of absorbent article 500 is illustrated. Elastic portion may be include traditional elastic stands in the belly area of absorbent article 500 (such as protective underwear) in such a manner to provide varying degrees of tension along the belly region 522. For example, elastic members 525a, 525b may include four elastic strands 70al 7b with 620 dtex to provide 225% elongation in a direction of the waist opening 2. It is contemplated that the elastic strands may be spaced apart from each other at a distance of about 0.25 to 0.5 inch to provide different the 2250% elongation. Alternatively, varying the dtex of the elastic stands or the number of strands may provide the desired elongation for the specific regions on the absorbent article 500. For example, top 20 mm of elastic member 525a may be above standard belly elastics of the absorbent article 500 and the elastic decitex may range from 540 to 940 dtex (or as desired for the specific design). It is contemplated that other spacing distances and decitex may be used. The benefits of such design include improved fit, greater feeling of security, and reduced instance of leakage for a wearer.

It is contemplated that any number of elastic regions may be provided on the absorbent articled 500 to alternative between regions of high or low tension. For example, as shown in FIG. 7, the chassis region 527 includes five elastic strands 80, thereby provided a region of higher tension than elastic members 525a, 525b. It is contemplated that varying the number of elastic strands 70a, 70b, 80, distance between the strands 70a, 70b, 80, and/or decitex of the strands 70a, 70b, 80 can provide for different regions of tension. The tension areas may or may not be of equal number of elastic strands 70a, 70b, 80, elastic weight % (decitex), or spacing. Furthermore, belly region 522 can alternate between regions of high tension and low tension to provide a greater sense of security of the absorbent article 500 when worn. For example, a portion of belly region 522 including five lycra strands at 800 dtex and 0.5 inch spacing between strands may provide 340% elongation and an adjacent portion of belly region 522 including four lycra strands at 620 dtex and 0.4 inch spacing may provide 300% elongation. It is contemplated that the elastic members 70a, 70b, 80 are generally made of non-woven material, but can also be made of woven stretch material.

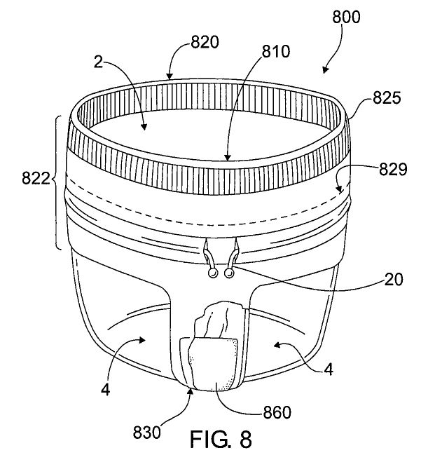

Referring now to FIG. 8, an absorbent article 800 according another exemplary embodiment is illustrated. The absorbent article 800 includes means for reducing the rise of the chassis on the wearer. The rise reducing means is optionally defined in the belly region of the chassis at a location between the waist opening and the tensioner, but other locations are contemplated as well. Various rise reducing means are contemplated, and suitable structures for reducing the rise of the chassis on the wearer include perforations, fold lines, discontinuities, areas of weakness, printed or otherwise applied indicia, and other structures that facilitate the reduction of the rise of the chassis by removing a portion of the chassis or by permanently or temporarily deforming the chassis.

Referring to the embodiment of absorbent article 800 illustrated in FIG. 8, the rise reducing means comprises perforations 829 extending substantially parallel to the waist opening 2 to reduce the rise of the chassis on the wearer. The perforations 829 may be defined in the belly region 822 of the chassis at a location between the waist opening 2 and the tensioner 20. When absorbent article 800 is worn by a wearer, an upper portion of the belly region 822 may rise over the waist of the wearer and the upper portion may be separated from the absorbent article 800 as desired by the wearer to provide a second rise, lower than the first rise to increase comfort for the wearer.

Alternatively, and according to another embodiment, the upper portion of the belly region 822 may be folded over so that elastic member 825 is below tensioner 20 to minimize the rise of the chassis. Advantages of an absorbent article 800 having perforations 829 and/or a fold line includes increased comfort and improved fit for a wearer.

Although the present invention has been particularly described in conjunction with specific embodiments, it is evident that many alternatives, modifications, and variations will be apparent to those skilled in the art. It is therefore contemplated that the appended claims will embrace any such alternatives, modifications, and variations as falling within the true scope and spirit of the present invention.

What is claimed:

1. An absorbent article comprising:
a chassis having a front section, a rear section, and a crotch section extending between the front section and the rear section, the front section being connected to the rear section to define a waist opening, leg openings, and a belly region positioned between the waist opening and the leg openings;
an absorbent core coupled to the crotch section of the chassis; and
an adjustable tensioner coupled to the chassis and positioned for snugging the chassis against a wearer of the absorbent article, wherein the tensioner is configured to be adjusted by the wearer from a first tension to a second, selected tension, wherein the first tension is less than the second, selected tension.

2. The absorbent article of claim 1, wherein the tensioner is coupled to the belly region of the chassis.

3. The absorbent article of claim 2, wherein the tensioner is coupled to a waist portion of the chassis.

4. The absorbent article of claim 2, wherein the tensioner is coupled to a side portion of the chassis.

5. The absorbent article of claim 2, wherein the tensioner is coupled to the leg openings of the chassis.

6. The absorbent article of claim 1, wherein the tensioner comprises drawstring.

7. The absorbent article of claim 6, wherein the drawstring comprises a single loop.

8. The absorbent article of claim 6, wherein the drawstring comprises a double loop.

9. The absorbent article of claim 1, further comprising at least two tensioners.

10. The absorbent article of claim 1, further comprising an elastic portion coupled to the chassis.

11. The absorbent article of claim 10, wherein the elastic portion is coupled to a belly region of the chassis.

12. The absorbent article of claim 10, wherein the elastic portion is coupled to a waist portion of the chassis.

13. The absorbent article of claim 10, wherein the elastic portion is coupled to leg openings of the chassis.

14. An absorbent article comprising:
a chassis having a front section, a rear section, and a crotch section extending between the front section and the rear section, the front section being connected to the rear section to define a waist opening, leg openings, a belly region positioned between the waist opening and the leg openings;
an absorbent core coupled to the crotch section of the chassis; and
at least one elastic member coupled to each of the waist opening and the belly region, the elastic members being spaced apart from each other and being stretchable independent of one another;
the elastic member coupled to the belly region having at least two elastic portions, wherein the tension in one of the elastic portions differs from the tension in another one of the elastic portions.

15. The absorbent article of claim 14, further comprising a chassis region positioned between the elastic members, the chassis region being stretchable in at least a direction along the waist opening.

16. The absorbent article of claim 14, wherein each of the elastic members comprises a non-woven material.

17. The absorbent article of claim 14, wherein each of the elastic members comprises at least one elastic strand.

18. The absorbent article of claim 17, wherein the at least one elastic strand has a density of about 540 decitex to about 940 decitex.

19. The absorbent article of claim 17, wherein the at least one elastic strand is stretchable in at least a direction along the waist opening to a length of about 200% elongation to about 350% elongation.

20. The absorbent article of claim 14, wherein the at least one elastic member comprises at least two elastic strands, each elastic strand being spaced apart from one another a distance from about 0.25 inch to about 0.5 inch.

21. An absorbent article comprising:
a chassis having a front section, a rear section, and a crotch section extending between the front section and the rear section, the front section being connected to the rear section to define a waist opening, leg openings, a belly region positioned between the waist opening and the leg openings, and a rise extending from the leg openings to the waist opening;
an absorbent core coupled to the crotch section of the chassis;
a tensioner coupled to the chassis and positioned for snugging the chassis against a wearer of the absorbent article; and
means for reducing the rise of the chassis on the wearer defined in the belly region of the chassis at a location between the waist opening and the tensioner.

22. The absorbent article of claim 21, wherein the rise reducing means comprises a fold line extending substantially parallel to the waist opening between the waist opening and the tensioner.

23. The absorbent article of claim 21, wherein the rise reducing means comprises perforations extending substantially parallel to the waist opening between the waist opening and the tensioner.

24. A method of manufacturing an absorbent article comprising the steps of:
attaching an absorbent core to a chassis having a contiguous front, rear, and crotch sections, the crotch section extending between the front section and the rear section; and
attaching a tensioner to the chassis such that the tensioner is adjustable by the wearer from a first tension to a second, selected tension, wherein the first tension is less than the second, selected tension.

25. The method of claim 24, further comprising attaching an elastic member to a waist portion of the chassis and orienting the elastic member such that the elastic member is stretchable in at least a direction of the waist opening.

26. The method of claim 24, further comprising attaching an elastic portion to leg portions of the chassis.

27. A method of manufacturing an absorbent article comprising the steps of:
attaching an absorbent core to a crotch section of a chassis; and
coupling at least one elastic member to each of a waist opening and a belly region of the chassis and spacing the elastic members from each other such that the elastic members are stretchable independent of one another;
configuring the elastic member coupled to the belly region to have at least two elastic portions such that the tension in one of the elastic portions differs from the tension in another one of the elastic portions.

28. A method of using an absorbent article, the method comprising the steps of:
positioning the absorbent article on a wearer; and
adjusting at least one adjustable tensioner of the absorbent article, thereby snugging the fit of the absorbent article on the wearer from a first tension to a second, selected tension, wherein the first tension is less than the second, selected tension.

29. The method according to claim 28, further comprising the step of folding a first waist portion or a belly region of the absorbent article over a second waist portion or a belly region of the absorbent article to reduce the rise of the absorbent article on the wearer from a first rise to a second, selected rise, wherein the second, selected rise is smaller than the first rise.

30. The method according to claim 28, further comprising the step of separating a portion of the absorbent article along a perforation to reduce the rise of the absorbent article on the wearer from a first rise to a second, selected rise, wherein the second, selected rise is smaller than the first rise.

31. The absorbent article of claim 1, wherein the absorbent article is selected from a group consisting of training pants, protective underwear, and adult diapers.

32. The absorbent article of claim 31, wherein the absorbent article is protective underwear.
